(19) United States
(12) Patent Application Publication
(10) Pub. No.: US 2006/0247265 A1
(43) Pub. Date: Nov. 2, 2006

(54) Therapies for treating disorders of the eye

(76) Inventors: Timothy P. Clackson, Arlington, MA (US); Leonard W. Rozamus, Bedford, MA (US)

Correspondence Address: ARIAD Gene Therapeutics, Inc., 26 Landsdowne Street, Cambridge, MA, 02139-4234, US

(21) Appl. No.: 11/116,942

(22) Filed: Apr. 28, 2005

(51) Int. Cl. A61K 31/4745

(52) U.S. Cl. 514/291

(57) ABSTRACT

Disclosed is the use of an mTOR inhibitor for treatment of certain eye disorders.

### BACKGROUND OF THE INVENTION

Despite the advent over the past decades of new drug treatments for a wide variety of illnesses, new drug treatments are still needed for a number of serious eye diseases, some of which are still considered untreatable. Others are treatable only with surgery and/or ophthalmic devices.

Eye disorders include, among others, disorders involving pathological neovascularization, ectopic proliferation, atrophy and nerve cell death, inflammation, infection and detachment. While a variety of approaches are known for treating inflammatory eye diseases (such as uveitis) and infections, new drug therapies are needed for treating or preventing other serious eye diseases.

Eye diseases of particular continuing concern, and which are the subject of this invention, including macular degeneration, certain retinopathies, neovascular glaucoma, retinal vein occlusion, and certain conditions arising from various ocular insults.

A variety of new therapeutic approaches have been suggested for such cases, including gene therapy, photodynamic therapy, and the use of antibodies, aptamers and matrix metalloprotease inhibitors, among others, although none of these have yet been proven efficacious and been approved for sale in the US. In view of the seriousness of the eye disorders addressed by this invention, new therapeutic methods, preferably using a small molecule drug such as disclosed herein, would clearly be of great benefit.

### SUMMARY OF THE INVENTION

This invention provides a new pharmaceutical method for treating or preventing age-related macular degeneration (“wet” or “dry” ARMD), neovascular glaucoma, retinopathy of prematurity, sickle-cell retinopathy, retinal vein occlusion, oxygen induced retinopathy, diabetic retinopathy, diabetic macular edema and macular edema associated with retinal vein occlusion and neovascularization due to ocular insults such as traumatic or surgical injury or transplantation of eye tissue in patients in need thereof, including patients suffering from such a disorder as well as those at risk thereof.

The method involves administering to the patient a treatment effective amount of an mTOR inhibitor such as rapamycin or one of its analogs or derivatives (“rapalogs”) or a prodrug thereof.

A variety of mTOR inhibitors are known in the art and include rapamycin and its C43 esters, ethers, carbamates, phosphinates and phosphonates (particularly aliphatic esters, ethers, carbamates, phosphinates and phosphonates such as Wyeth's CCI-779, Novartis' Everolimus and ARIAD's AP23573); C43 tetrazole derivatives such as Abbott's ABT-578; other C43 phosphorous-containing derivatives such as are disclosed in PCT/US03/03030; as wells as derivatives of any of the foregoing having one or more of the following modifications: epimerization at C43, epimerization at C28, and replacement of the 7-methoxyl group with a hydroxyl or with H. Additional other mTOR inhibitors have been previously disclosed in the patent and scientific literature.

The administration of the mTOR inhibitor may be systemic (e.g., parenteral or oral) or local, with local delivery directly to the eye being of particular interest.

The effective dose will typically be in the range of about 0.01 to about 100 mg of mTOR inhibitor/kg body weight of patient, preferably about 0.1 to about 10 mg/kg of mammalian body weight, administered in single or multiple doses. Generally, the compound may be administered in a daily dose range of about 1 to about 2000 mg per patient. Administration may be once or multiple times daily, weekly (or at some other multiple-day interval) or on an intermittent schedule such as disclosed in WO 03/064383. When administered by injection into the eye, less frequent administration and doses at the lower end of the ranges will usually suffice. Topical application to the eye will generally involve relatively lower doses, though not necessarily less frequent.

A variety of formulations appropriate for the different routes of administration are known for rapamycin itself and for a variety of rapalogs and may be adapted to the practice of this invention. Typically a composition is prepared which contains the drug and one or more pharmaceutically acceptable diluents or excipients. Local administration to the eye is of particular interest and may be achieved by injection, by topical application, or by application into or onto the eye of a device such as a contact lens or other support material which contains the mTOR inhibitor and following application to the eye releases the drug into the eye.

The mTOR inhibitor may be administered as a monotherapy—i.e., as a single agent, not in combination with other drugs (small molecules, proteins, antibodies, antisense molecules, siRNA or expressible genes, as in the case of gene therapy) for treating the eye disorder. For the purpose of this document, the administration is considered a monotherapy/single agent application so long as any other pharmacologically active agents administered in conjunction with the mTOR inhibitor are administered for ancillary reasons (local anesthetic, antibiotic, enhancement of absorption or penetration, etc.) rather than to treat the disease. Alternatively, the mTOR inhibitor may be administered in combination with another pharmacologically active agent chosen from a small molecule drug (i.e., a drug other than a protein or nucleic acid and which has a molecular weight under 2000 molecular mass units, preferably under 1200, and typically under 750), antibody or antibody fragment, protein therapeutic agent, aptamer, antisense molecule, siRNA molecule or an analog, derivative or prodrug of any of the foregoing.

The method of this invention is of particular interest for the treatment of forms of macular degeneration and diabetic retinopathy.

mTOR Inhibitors

Rapamycin is a macrolide antibiotic produced by Streptomyces hygroscopicus. It binds to a FK506-binding protein, FKBP12, with high affinity to form a rapamycin:FKBP complex. Reported Kd values for that interaction are as low as 200 pM. The rapamycin:FKBP complex binds with high affinity to the large cellular protein, FRAP, to form a tripartite, [FKBP:rapamycin]:[FRAP], complex. In that complex rapamycin can be viewed as a dimerizer or adapter to join FKBP to FRAP (which is also referred to as “mTOR”). Formation of the complex is associated with rapamycin's various biological activities.

This document adopts the rapamycin numbering convention illustrated on page 1 of WO 01/14387 in which a hydroxyl group is attached to rapamycin's cyclohexyl ring at carbon atom 43 (“C43”):

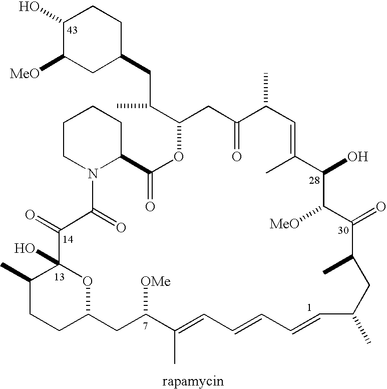

Rapamycin is a potent immunosuppressive agent and is used clinically to prevent rejection of transplanted organs. It is known to have a number of additional pharmacological activities. Certain mTOR inhibitors, including rapamycin and/or its analogs, AP23573 (ARIAD Pharmaceuticals, Inc., see WO 03/064383, Example 9), CCI779 (Wyeth, 3-hydroxy-2-(hydroxymethyl)-2-methylproprionic acid ester or rapamycin at C43; see WO 02/40000 and U.S. Pat. No. 5,362,718), SDZ Rad (“RAD001” or “Everolimus” Novartis; hydroxyethyl ether at C43) and ABT-578 (Abbott; tetrazole at C43, see WO 99/15530) are promising agents for treating certain cancers, for immune suppression and/or for helping to decrease the incidence of restenosis following interventional cardiology. See e.g. published US Patent application 2001/0010920 and WO 02/098416 and patent and literature references cited therein.

Rapamycin's pharmacological potential has stimulated the search for rapamycin analogs with improved therapeutic index, pharmacokinetics, formulatability, ease or economy of production, etc. The resulting investigation by the pharmaceutical industry and academic researchers has generated an extensive literature on materials and methods for effecting chemical transformations of rapamycin, including reductions of ketones, demethylations, epimerizations, various acylations and alkylations of hydroxyls, etc.

Many structural variants of rapamycin have now been reported, typically arising as alternative fermentation products and/or from synthetic efforts. For example, the extensive literature on analogs, homologs, other derivatives and other compounds related structurally to rapamycin include, among others, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Additional historical information is presented in the background sections of U.S. Pat. Nos. 5,525,610; 5,310,903 and 5,362,718. See also U.S. Pat. No. 5,527,907 and WO 02/080975. Materials and methods have even been developed for the remarkably effective and selective epimerization of the C-28 hydroxyl group (WO 01/14387). Additional rapamycin analogs are disclosed in International Patent Application PCT/US03/03030 and U.S. patent application Ser. No. 10/357152, the full contents of both of which are incorporated herein by reference. Rapamycin and its analogs can also be prepared such that they incorporate one or more atoms of less common isotopes of one or more of H, C, N or O, e.g., substituting deuterium for one more more occurrences of H, for instance.

The mTOR inhibitor used in the practice of this invention may be rapamycin or any of its pharmacologically active analogs or derivatives, including among others, 43-epi-rapamycin, variants which are alkylated or acylated at position 43 such as CCI 779 and RAD 001, the rapamycin analogs disclosed in PCT/US03/03030, or 7-desmethyl or 7-desmethoxyl variants of any of the foregoing, and the variety of other rapamycin analogs disclosed in the references cited herein, or a pharmaceutically acceptable derivative of any of the foregoing. The mTOR inhibitor, if other than rapamycin, AP23573, CCI779, RAD001 or ABT578, should preferably (a) retain at least 0.01, preferably 0.1 and more preferably at least 0.5 times the potency of rapamycin in any conventional T cell proliferation assay; or (b) have at least 0.1 times the biological half life of rapamycin (if not an extended biological half life) as measured in any conventional rodent or primate based assay. Preferably the compound meets both of the foregoing potency and half life criteria. Alternatively, the compound may be a prodrug of an mTOR inhibitor, which upon administration to a patient, is converted in vivo to yield an mTOR inhibitor which meets one or both of the foregoing criteria.

While rapamycin has been suggested for use in treating inflammatory eye disease such as uveitis, neither it nor any other mTOR inhibitor have heretofore been disclosed for treating the quite different eye disorders addressed by the present invention.

Formulations, Pharmaceutical Compositions, Dosage and Administration

The mTOR inhibitor may be formulated using materials and methods based on those known for rapamycin or its analogs or, particularly in the case of local delivery to the eye, may be based on formulations developed for other ophthalmic pharmaceuticals. For instance, solutions, suspensions, emulsions, pills, tablets and other formulations based on micro emulsions, nanosizing or solid dispersions, as well as a variety of carriers, stabilizing, time delaying, wetting, dispersing and other excipients, are known for rapamycin or its analogs which may be adapted to the practice of this invention. See e.g., WO 064383, especially pages 31-32 and 63-69, and references cited therein.

The mTOR inhibitor may be administered systemically in any manner useful in directing the active compounds to the recipient's bloodstream or site of action, including orally, parenterally (including intravenous, intramusculart intraperitoneal and subcutaneous injections as well as injection into the eye), via implants, rectally, intranasally, vaginally, and transdermally. For the purposes of this disclosure, transdermal administrations are understood to include all administrations across the surface of the body and the inner linings of bodily passages including epithelial and mucosal tissues. Such administration may be carried out using the mTOR inhibitor, or pharmaceutically acceptable salts or prodrugs thereof, in lotions, creams, foams, patches, suspensions, solutions, and suppositories (rectal and vaginal).

Various delivery systems are known and can be used to administer the compound, or the various formulations thereof, including tablets, capsules, injectable solutions, encapsulation in liposomes, microparticles, microcapsules, etc. Methods of introduction include but are not limited to dermal, intradermal, intramuscular, intraperitoneal, intravenous, subcutaneous, intranasal, pulmonary, epidural, ocular and oral routes. The compound may be administered by any convenient or otherwise appropriate route, for example by infusion or bolus injection, by absorption through epithelial or mucocutaneous linings (e.g., oral mucosa, rectal and intestinal mucosa, etc).

In certain embodiments, it may be desirable to administer the mTOR inhibitor locally using an implant typically being of a porous, non-porous, or gelatinous material, including membranes, such as sialastic membranes or fibers or other support or matrix materials bearing the mTOR inhibitor.

For example, a solution of the mTOR inhibitor for injection may contain 0.1 to 10 mg/ml, e.g. 1-3 mg/ml, of rapalog in a diluant solution containing Phosal 50 PG (phosphatidylcholine, propylene glycol, mono- and di-glycerides, ethanol, soy fatty acids and ascorbyl palmitate) and polysorbate 80, containing 0.5-4% ethanol, e.g. 1.5%-2.5% ethanol. As another example, the diluant may contain 2-8%, e.g. 5-6%, each of propylene glycol USP and polysorbate 80 in water for injection. We have found that 5.2% of each works well in some cases. Typically a solution is processed using conventional methods and materials, including e.g. one or more rounds of sterile filteration.

Again, materials and methods for producing the various formulations are known in the art and may be adapted for practicing the subject invention. See e.g. U.S. Pat. Nos. 5,182,293 and 4,837,311 (tablets, capsules and other oral formulations as well as intravenous formulations) and European Patent Application Publication Nos. 0 649 659 (published Apr. 26, 1995; illustrative formulation for IV administration) and 0 648 494 (published Apr. 19, 1995; illustrative formulation for oral administration). See also U.S. Pat. No. 5,145,684 (nanoparticles) and U.S. Pat. No. 5,989,591 (solid dosage forms) and WO 98/56358 as well as Yu, K. et al., Endocrine-Related Cancer (2001) 8, 249-258 and Geoerger et al., Cancer Res. (2001) 61 1527-1532.

The amount of compound which will be effective in the treatment or prevention of a particular disorder or condition will depend in part on well known factors affecting drug dosage such as the particular compound utilized, the mode of administration, the condition, and severity thereof, of the condition being treated, as well as the various physical factors related to the individual being treated. In addition, in vitro or in vivo assays may optionally be employed to help identify optimal dosage ranges. Effective doses may be extrapolated from dose-response curves derived from in vitro or animal model test systems. The precise dosage level should be determined by the attending physician or other health care provider and will depend upon well known factors, including route of administration, and the age, body weight, sex and general health of the individual; the nature, severity and clinical stage of the disease; and the use (or not) of concomitant therapies.

In many cases, satisfactory results may be obtained when the mTOR inhibitor is administered systemically in a daily dosage of from about 0.01 mg/kg-100 mg/kg, preferably between 0.01-25 mg/kg, and more preferably between 0.01-5 mg/kg. The projected daily dosages are expected to vary with route of administration. Thus, parenteral dosing will often be at levels of roughly 10% to 20% of oral dosing levels.

In cases of topical administration to the eye, e.g., in the case of eye drops, the composition containing the mTOR inhibitor (typically as a solution or suspension) may also include viscosity enhancing materials, usually polymers (e.g., poly(vinyl alcohol), poly(vinylpyrrolidone) and various cellulose derivatives. In some cases the viscosity enhancing materials are able to interact with the mucous layer on the eye surface or can transform from a solution to a gel under the conditions present at the pre-ocular area. A viscosity in the range of from about 1,000 to 30,000 centipoise is generally considered useful for a drop. About 30,000 to about 100,000 centipoise is an advantageous viscosity range for ophthalmic administration in ribbon form. The viscosity can be controlled in many ways as is well known in the art. The ophthalmic compositions may contain one or more of the following: surfactants, adjuvants including additional medicaments, buffers, antioxidants, tonicity adjusters, preservatives, thickeners or viscosity modifiers, and the like. Additives in the formulation may desirably include sodium chloride, EDTA (disodium edetate), and/or BAK (benzalkonium chloride), sorbic acid, methyl paraben, propyl paraben, chlorhexidine, and sodium perborate. Cyclodextrins may also be included in the composition to aid in penetration and absorption. See e.g., Davies, Clinical and Exp. Pharmacology and Physiology (2000) 27, 558-562; Loftsson tet al, Acta Ophthalmologica Scand 2002:80:144-150; and references cited in both. Additional excipients which may be included with the mTOR inhibitor in compositions for topical delivery to the eye are disclosed in WO 01/68053 and U.S. Pat. No. 6,569,443. Such compositions for topical delivery to the eye may contain the mTOR inhibitor in an amount from 0.01 to 10% wt/wt, in some cases 0.05 to 5.0%, and in others from 0.1 to 1%.

The mTOR inhibitor may also be administered using a drug-bearing hydrogel contact lens which gradually releases the drug after application to the eye. For instance, hydroxyethyl methacrylate can be combined with ethylene glycol dimethacrylate to generate a hdrogel from which dissolved oxygen can be removed using a nitrogen purge. A solution or microemulsion of the mTOR inhibitor or a suspension of nanoparticles (no bigger than about 100 nm) of the drug, for example, is then added. Azo-bis-iso-butrylonitrile is added to the solution and dissolved. The solution is placed between a mold made of two glass plates and polymerized in a 60° C. oven for approximately 22 hours. The sample gel formed is 1 millimeter in thickness. Alternatively, an aqueous solution or suspension of the mTOR inhibitor may simply be loaded on preformed hydrogel lenses. The lenses are inserted into the eye where the mTOR inhibitor is eluted over time. Each lens is eventually discarded and replaced with a new drug-bearing lens as needed to deliver the appropriate amount of drug.

When the mTOR inhibitor is used as part of a combination regimen, dosages of each of the components of the combination are administered during a desired treatment period. The components of the combination may administered at the same time; either as a unitary dosage form containing both components, or as separate dosage units; the components of the combination can also be administered at different times during a treatment period, or one may be administered as a pretreatment for the other.

The invention also provides a pharmaceutical pack or kit comprising one or more containers containing one or more of the ingredients of the pharmaceutical compositions of the invention. Optionally associated with such container(s) can be a notice in the form prescribed by a governmental agency regulating the manufacture, use or sale of pharmaceutical or biological products, which notice reflects approval by the agency of manufacture, use or sale for human administration. The notice or package insert may contain instructions for use of a rapalog of this invention, consistent with the disclosure herein.

The following examples contain important additional information, exemplification and guidance which can be adapted to the practice of this invention in its various embodiments and the equivalents thereof The examples are offered by way illustration should not be construed as limiting in any way. Numerous modifications and variations of the present invention should be apparent to one of skill in the art. Such modifications and variations, including choices in selecting, preparing, formulating and administering the mTOR inhibitor and are intended to be encompassed by the scope of the invention and of the appended claims.

The contents of all cited references including literature references, issued patents, and published patent applications as cited throughout this document are hereby expressly incorporated by reference. The practice of the present invention will employ, unless otherwise indicated, conventional techniques of ophthalmic care, drug formulation and administration, which are within the skill of the art. Such techniques are explained fully in the patent and scientific literature.

### EXAMPLES

### Example 1

### Administration of an mTOR Inhibitor by Injection into the Eye

A numbing eye drop, an antibiotic eye drop, and an injected antibiotic are first administered to the eye. The solution of 1-20 mg of AP23573 is injected into the eye's vitreous. After the injection, the patient lies on his or her back for 30 minutes. An antibiotic eye ointment is used for 1-7 days following treatment. Treatment is repeated as necessary every on e- three months.

### Example 2

### Administration of an mTOR Inhibitor by Injection into the Eye

Patients receive AP23573 injections through a needle into the eye's vitreous. Six injections of 1-25 mg are given over a 30-week period. Before each injection, the surface of the eye is numbed with anesthetic eye drops. This is followed by injection of another anesthetic into the lower portion of they eye in the clear tissue surrounding the white of the eye. After a few minutes, the AP23573 is injected into the vitreous. Patients receive AP23573 injections once every two-six weeks, to be continued as needed.

1. A method for treating age-related macular degeneration, neovascular glaucoma, retinopathy of prematurity, sickle-cell retinopathy, retinal vein occlusion, oxygen induced retinopathy, diabetic retinopathy, diabetic macular edema, macular edema associated with retinal vein occlusion and neovascularization due to ocular insults such as traumatic or surgical injury or transplantation of eye tissue in patients in need thereof, the method comprising administering to the patient a treatment effective amount of an mTOR inhibitor.

2. The method of claim 1 wherein the mTOR inhibitor is rapamycin, CCI-779, RAD001, ABT-578 or AP23573.

3. The method of any of claims 1 or 2 wherein the administered is effected by administering to the patient a composition containing the mTOR inhibitor and one or more pharmaceutically acceptable diluents or excipients.

4. The method of any of claims 1-3, wherein the administration is oral.

5. The method of any of claims 1-3, wherein the administration is parenteral.

6. The method of any of claims 1-3, wherein the administration is local administration to the eye.

7. The method of claim 6, wherein the administration comprises insertion of a device which elutes an mTOR inhibitor.

8. The method of claim 7, wherein the disease is a contact lens which elutes an mTORinhibitor.

9. The method of any of claims 1-6, wherein the disease is macular degeneration.

10. The method of any of claims 1-6, wherein the disease is diabetic retinopathy.
